(19) United States
(12) Patent Application Publication
(10) Pub. No.: US 2009/0111836 A1
(43) Pub. Date: Apr. 30, 2009

(54) Angular Pyranocoumarins, Process for Preparation and Uses Thereof

(75) Inventors: Wangfun Fong, Hong Kong (HK); Xiaoling Shen, Guangzhou (CN); Guangying Chen, Haikou (CN); Guoyuan Zhu, Shenzhen (CN); Chikeung Wan, Hong Kong (HK); Kaiwing Tse, Hong Kong (HK)

Correspondence Address: MCDONNELL BOEHNEN HULBERT & BERGHOFF LLP, 300 S. WACKER DRIVE, 32ND FLOOR, CHICAGO, IL, 60606, US

(73) Assignee: CITY UNIVERSITY OF HONG KONG

(21) Appl. No.: 11/931,455

(22) Filed: Oct. 31, 2007

(51) Int. Cl. A61K 31/352; C07D 493/02; A61P 35/04; A61K 31/439; A61K 31/522

(52) U.S. Cl. 514/263.23; 549/387; 514/455; 514/283

(57) ABSTRACT

The present invention relates to compounds, compositions for use in reversing multidrug resistance in cancer cells, process for the preparation thereof and their uses in treating cancers. More particularly, the present invention relates to 3′,4′-aromatic acyloxy substituted 7,8-pyranocoumarins compounds for use in reversing P-glycoprotein overexpression mediated multidrug resistance in cancer cells, pyranocoumarins containing compositions, process for the preparation thereof and their uses in treating cancers.

### FIELD OF THE INVENTION

The present invention relates to compounds, compositions for use in reversing multidrug resistance in cancer cells, process for the preparation thereof and their uses in treating cancers. More particularly, the present invention relates to 3′,4′-aromatic acyloxy substituted 7,8-pyranocoumarin compounds for use in reversing P-glycoprotein over-expression mediated multidrug resistance in cancer cells, process for the preparation thereof, pyranocoumarins containing composition, and their uses in treating cancers.

### BACKGROUND ART

Symptom of multidrug resistance (MDR) is generally reported in cancer patients in the course of their chemotherapy treatment, of which partial cancer cells are still allowed to survive and keep growing under the influence of a single anticancer drug, and show resistance to a wide spectrum of structurally and functionally unrelated anti-cancer agents, which results in the reduction of chemotherapy efficacy or even leads to chemotherapy failure.

A number of mechanisms have been described to explain the phenomenon of MDR in mammalian cells (1. Krishna R, Mayer L D. Multidrug resistance (MDR) in cancer. Mechanisms, reversal using modulators of MDR and the role of MDR modulators in influencing the pharmacokinetics of anticancer drugs. Eur J Pharm Sci 2000; 11:265-283. 2. Stavrovskaya A A. Cellular mechanisms of multidrug resistance of tumor cells. Biochemistry (Moscow) 2000; 65:95-106. 3. Ozben T. Mechanisms and strategies to overcome multiple drug resistance in cancer. FEBS Lett 2006; 580:2903-9). These include Glutathione S-transferease (GST) overexpression, which enhances metabolic biotransformation of many anticancer drugs or xenobiotic detoxification; upregulation of DNA topoisomerase II or topoisomerase II gene mutation, which neutralize actions of anticancer drugs targeting at topoisomerase II; mutation of tumor suppressor gene p53 that deregulates cell cycle arrest in G₁ and apoptosis following DNA damage caused by anticancer drugs, or overexpression of bcl-2, a gene that block cell death; overexpression of lung-resistance-related protein (LRP) in the cytoplasm which participate in the transport of substrates from nucleus to cytoplasm and sequestration into vesicles; lastly, overexpression of ATP-binding cassette (ABC) transporters such as multidrug resistance associated protein (MRP), P-glycoprotein (P-gp) and breast cancer resistance protein (BCRP) that cause reduced intracellular drug accumulation through binding with anticancer drug substrates and bringing them out of cells. Of all these mechanisms, P-gp-directed drug transport has been studied in most detail and appears to be a very common mechanism of MDR both in vitro and vivo.

P-gp is an ATP-dependent plasma membrane transporter protein encoded by MDR1 gene. P-gp is expressed with high level in the tissues of liver, gastrointestinal mucous membrane, kidney and pancreas etc, and is proposed to function as an efflux pump, excreting xenotoxins from the membrane bilayer to the exterior, therefore preventing body from damage by exogenous substances from food, drugs or environment. Many currently used chemotherapeutic anticancer drugs are P-gp substrates. These drugs are mainly structurally and functionally unrelated hydrophobic or amphipathic natural products, including anthracyclines, vinca alkaloids, taxanes, and podophyllotoxins (1. Krishna R, Mayer L D. Multidrug resistance (MDR) in cancer. Mechanisms, reversal using modulators of MDR and the role of MDR modulators in influencing the pharmacokinetics of anticancer drugs. Eur J Pharm Sci 2000; 11:265-283. 2. Ambudkar S V, Kimchi-Sarfaty C, Sauna A U, Gottesman M M. P-glycoprotein: from genomics to mechanism. Oncogene, 2003; 22:7468-7485. 3. Ozben T. Mechanisms and strategies to overcome multiple drug resistance in cancer. FEBS Lett 2006; 580:2903-9). Over-expression of P-gp in tumor tissues can greatly decrease substrate drug accumulation within tumor cells and cause failure of chemotherapy because P-gp actively pumps them out by using ATP.

Resistance of tumor to an anticancer agent can be reversed by coadminstering a multidrug resistance modulator (or inhibitor) with the anticancer agent. P-gp modulators themselves are non-toxic compounds or compounds with low toxicity, with no effect on cell proliferation, but can increase cellular accumulation of anticancer drugs that are P-gp substrates through inhibiting P-gp-mediated drug efflux, therefore enhance or restore drug sensitivity of MDR cells. Said P-gp modulator includes Verapamil (a coronary artery dilating drug), Reserpine (an antihypertensive drug), Cyclosporin A (immunosuppressant), XR9576, PSC-833, LY-335979, VX-710 and the like. In general, these drugs or compounds are small hydrophobic aromatic molecules, which can bind to P-gp in a competitive, or non-competitive manner so as to inhibit the transportation of anti-cancer medicaments by P-gp. Though several candidates such as XR9576, LY335979 are undergoing phase III clinical trial, there are currently no clinically applicable P-glycoprotein modulators (1. Kohler S, Stein W D. Optimizing chemotheraphy by measuring reversal of P-glycoprotein activity in plasma membrane vesicles. Biotechnol Bioeng 2003; 81:507-517. 2. Dantzig A H, Shepard R L, Cao J, Law K L, Ehlhardt W J, Baughman T M, Bumol T F, Starling J J. Reversal of P-glycoprotein mediated multidrug resistance by a potent cyclopropyldibenzosuberane modulator LY 335979. Cancer Res 1996; 56:4171-4179. 3. Ozben T. Mechanisms and strategies to overcome multiple drug resistance in cancer. FEBS Lett 2006; 580:2903-9). Accordingly, development of an inhibitor specific to P-gp with low toxicity has become a hotspot of research and development among the scientists.

(±)-Praeruptorin A, isolated from a medicinal plant Peucedanum praeruptorum Dunn, is the first angular pyranocoumarin (also called 7,8-pyranocoumarin) discovered that increases drug sensitivity of Pgp-MDR cells, but its enhancement effect is only moderate (Wu J Y, Fong W F, Zhang J X, Leeung C H, Kwong H L, Yang M S, Li D, Cheung H Y. Reversal of multidrug resistance in cancer cells by pyranocoumarins isolated from Radix Peucedani. Eur J Pharmacol 2003; 473:9-17).

### SUMMARY OF THE INVENTION

In these circumstances, due to the limitations (significant toxic side effects) of the multidrug resistance reversing agents in the art, multidrug resistance reversing agents that definitely reverse the multidrug resistance caused by P-gp over-expression, increase the sensitivity of cancer cells to anti-cancer medicaments, and increase the therapeutic efficacy of anti-cancer medicaments are required.

Contribution to the above-mentioned problems is provided by the structural modification of (±)-Praeruptorin A by the inventor, which results in the formation of a series of derivatives, i.e. 3′,4′-aromatic acyloxy substituted 7,8-pyranocoumarin compounds. It was found that as compared with (±)-Praeruptorin A, 3′,4′-aromatic acyloxy substituted 7,8-pyranocoumarins possess lower toxicity and better activity in the reversal of multidrug resistance, which lead to the development of new multidrug resistance reversing agent for inhibiting cancer that exhibits higher activity and lower toxicity. In addition, the present invention discloses the process for the preparation and the use of the above compounds.

More particularly, the present invention provides 3′,4′-aromatic acyloxy substituted 7,8-pyranocoumarin compounds having the following general formula:

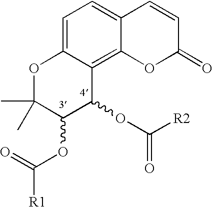

wherein, R1 and R2 may be the same or different, independently represents aryl, and ester groups at C-3′ and C-4′ are either in cis-configuration or in trans-configuration. Said cis-configuration may be 3′(R),4′(R) or 3′(S),4′(S) or combination of these two; trans-configuration may be 3′(R),4′(S) or 3′(S),4′(R) or combination of these two. Preferred aryl is selected from alkoxy,substituted aryl, and more preferably, aryl is selected from methoxy substituted aryl such as those selected from the group consisting of 4-methoxyphenyl, 4-methoxybenzyl, 4-methoxystyryl, 3,4-dimethoxyphenyl, 3,4-dimethoxybenzyl and 3,4-dimethoxystyryl. More preferably, compound of the present invention is (±)-3′-O,4′-O-bis(3,4-dimethoxybenzoyl)-cis-khellactone, (±)-3′-O,4′-O-bis(3,4-dimethoxybenzoyl)-trans-khellactone, (±)-3′-O,4′-O-bis(3,4-dimethoxycinnamoyl)-cis-khellactone or (±)-3′-O,4′-O-bis(3,4-dimethoxycinnamoyl)-trans-khellactone.

In another aspect, the present invention also provides the process for the preparation of 3′,4′-aromatic acyloxy substituted 7,8-pyranocoumarin compounds, which comprises the following steps:

(a) To prepare (±)-3′,4′-cis-dihydoxy-7,8-pyranocoumarin (also called (±)-cis-khellactone) and (±)-3′,4′-trans-dihydoxy-7,8-pyranocoumarin (also called (±)-trans-khellactone) respectively using (±)-Praeruptorin A as the lead compound; and

(b) To prepare (±)-3′,4′-cis-diaromatic acyloxy substituted 7,8-pyranocoumarins and (±)-3′,4′-trans-diaromatic acyloxy substituted 7,8-pyranocoumarins from (±)-cis-khellactone and (±)-trans-khellactone, respectively.

More particularly, the process of the present invention includes the following step (a) and (b):

(a) To dissolve (±)Praeruptorin A in dioxane and stir it at about 60° C. for 10˜30 minutes in the presence of about 0.5 M potassium hydroxide, followed by slow addition of about 10% sulphuiric acid at room temperature for acidification, and then extract the resultant reaction solution with chloroform, and undergo purification with silica gel column chromatography to afford (±)-cis-khellactone and (±)-trans-khellactone, respectively; and

(b) To dissolve (±)-cis-khellactone and (±)-trans-khellactone in dichloromethane, respectively, followed by stirring under reflux with 5˜8 times by mole of aromatic carboxylic acid compound in the presence of dicyclohexylcarbodiimide (DCC) and 4-dimethylamino pyridine (DMAP) for 2.5˜3 hours. Filter the resultant reaction solution after cooling, and the filtrate is subjected to purification with column chromatography to afford pure (±)-3′,4′-cis-diaromatic acyloxy substituted 7,8-pyranocoumarins and (±)-3′,4′-trans-diaromatic acyloxy substituted 7,8-pyranocoumarins, respectively.

In another aspect, the present invention also provides pharmaceutical compositions containing the compounds of the present invention. Said pharmaceutical compositions may be in the form of parenteral preparations or oral preparations, but not limited to these.

Preferred parenteral preparations according to the present invention are injection preparations, but not limited to these. Preferred oral preparations according to the present invention are tablets, capsules, granules and oral solution, but not limited to these.

It should be understood that compositions containing the compounds of the present invention can be formulated into the required preparations by the person skilled in the art according to the conventional pharmaceutical preparation process in the art.

In another aspect, the present invention also provides the method for treating cancers, which includes the step of administering therapeutically effective amount of the compounds of the present invention or pharmaceutical compositions of the present invention to the subjects in need thereof, wherein mammals are the preferred subjects, and human is more preferred. Preferably, said compounds or compositions possess the following efficacies:

(a) Increasing the sensitivity of multidrug-resistant cancer cells to anti-cancer medicaments;

(b) Reactivating Doxorubicin in drug-resistant cells to induce G2/M arrest, which lead to cells apoptosis;

(c) Significantly increasing drug accumulation level of Doxorubicin in drug-resistant cells; or

(d) Significantly decreasing the expulsion of Rh-123 and H33342 in drug,resistant cells.

Additionally, the compound or the pharmaceutical composition above mentioned may be administered to a subject in need thereof in combination with an anticancer medicament. The anti-cancer medicament includes but not limited to Doxorubicin, Vinblastine, Puromycin and/or Paclitaxel. The significant efficacy of the present invention is that 3′,4′-aromatic acyloxy substituted 7,8-pyranocoumarin compounds are effective in reversing multidrug resistance in cancer cells, which is superior to their precursor compound, (±)-Praeruptorin A, and that the process for the preparation thereof is simple and practicable. A drug that can definitely reverse the multidrug resistance and increase the therapeutic efficacy of anti-cancer medicaments is provided.

### BRIEF DESCRIPTION OF THE DRAWINGS

FIG. 1 shows that (±)-Praeruptorin A derivatives and Verapamil increase the accumulation level of Doxorubicin in K562-DR and HepG2-DR cells, wherein the increased cellular level of Doxorubicin (%)=100×(F_{S}−F₀)/F₀

(Fs: cellular fluorescence intensity of Doxorubicin in the presence of testing drugs; F₀: cellular fluorescence intensity of Doxorubicin in the absence of any P-gp modulator).

FIG. 2 shows that (±)-Praeruptorin A derivatives and Verapamil reduce the expulsion of Rh-123 in K562-DR and HepG2-DR cells, wherein the increased cellular level of Rh-123 (%)=100×(F_{S}−F₀)/F₀

(Fs: cellular fluorescence intensity of Rb-123 in the presence of testing drugs; Fo: cellular fluorescence intensity of Rh-123 in the absence of any P-gp modulator).

FIG. 3 shows that (±)-Praeruptorin A derivatives inhibit the expulsion of H33342 in HepG2-DR cells.

Relative amount of intracellular H33342 (%)=100×(F_{S}−F₀)/F₀

(Fs: cellular fluorescence intensity of H33342 in the presence of a testing drug; F₀: cellular fluorescence intensity of H33342 in the absence of any P-gp modulator).

FIG. 4 shows that (±)-Praeruptorin A derivatives do not affect the expression of P-gp in drug-resistant cells. After 72-hour incubation in a complete culture solution containing respectively 4 μM of cis-DMDCK (3), 4 μM of cis-DMDBK (4), 4 μM of trans-DMDCK (5) and 4 μM of trans-DMDBK (6), and in a complete culture solution in the absence of the above four components (2), changes in P-gp level in HepG2-DR, KB V1, K562-DR cells were not observed. (1) is the control for sensitive cells.

FIG. 5 shows the influence of (±)-Praeruptorin A derivatives on the binding of UIC2 to P-gp. Under the influences of 5 μM of cis-DMDCK, cis-DMDBK or trans-DMDBK, intracellular fluorescence intensity increases due to the increase in the binding of UIC2 to P-gp in HepG2-DR cells. Under the influences of 5 μM of trans-DMDCK, intracellular fluorescence intensity decreases due to the reduction of the binding of UIC2 to P-gp in HepG2-DR cells.

### DETAILED DESCRIPTION OF THE INVENTION

The 7,8-pyranocoumarins according to the present invention and the pharmacological activities thereof were prepared or discovered according to the examples shown below. Said preparation process employed in the present invention relates to technical means that the person skilled in the art can completely master and apply. However, the following examples should not be construed to limit the scope of the appended claims in meaning.

### EXAMPLE 1

### Preparation and Structure Identification of (±)-3′-O,4′-O-bis(3,4-dimethoxycinnamoyl)-cis-khellactone

(±)-cis-khellactone (80 mg, 0.31 mmol) was dissolved in 5 ml dichloromethane, followed by addition of 3,4-dimethoxycinnamic acid (310 mg, 1.5 mmol), DCC (206 mg, 1 mmol), DMAP (4 mg, 0.032 mmol), reaction was allowed to stir under reflux for 2.5˜3 hours, and then left it to cool. The filtrate obtained from filtration was subjected to purification with flash column chromatography on silica gel using mixed solvent of petroleum ether/ethyl acetate (75:25) as eluent. Fractions were monitored by liquid chromatography-mass spectrometry (LC/MS). Factions containing component with molecular weight of M=642 were collected, dried, and further purified by recrystallization in mixed solvent of petroleum ether and ethyl acetate to afford 22 mg of pure (±)-3′-O,4′-O-bis(3,4-dimethoxycinnamoyl)-cis-khellactone represented by cis-DMDCK, optical Rotation [(α]_{D}=0 (for its Proton Nuclear Magnetic Resonance Spectroscopy (¹H-NMR), see table 1).

### EXAMPLE 2

### Preparation and Structure Identification of (±)-3′-O,4′-O-bis(3,4-dimethoxybenzoyl)-cis-khellactone

(±)-cis-khellactone (80 mg, 0.3 mmol) was dissolved in 5 ml dichloromethane, followed by addition of 3,4-dimethoxybenzoic acid (270 mg, 1.5 mmol), DCC (206 mg, 1 mmol), DMAP (4 mg, 0.032 mmol), reaction was allowed to stir under reflux for 2.5˜3 hours, and then left it to cool. The filtrate obtained from filtration was subjected to purification with flash column chromatography on silica gel using mixed solvent of petroleum ether/ethyl acetate (75:25) as eluent. Fractions were monitored by LC/MS. Factions containing component with molecular weight of M=590 were collected, dried, and further purified by recrystallization in mixed solvent of petroleum ether and ethyl acetate to afford 40 mg of pure (±)-3′-O,4′-O-bis(3,4-dimethoxybenzoyl)-cis-khellactone represented by cis-DMDBK, optical Rotation [α]_{D}=0 (for its Proton Nuclear Magnetic Resonance Spectroscopy (¹H-NMR), see table 1).

### EXAMPLE 3

### Preparation and Structure Identification of (±)-3′-O,4′-O-bis(3,4-dimethoxycinnamoyl)-trans-khellactone

(±)-trans-khellactone (80 mg, 0.3 mmol) was dissolved in 5 ml dichloromethane, followed by addition of 3,4-dimethoxycinnamic acid (310 mg, 1.5 mmol), DCC (206 mg, 1 mmol), DMAP (4 mg, 0.032 mmol), reaction was allowed to stir under reflux for 2.5˜3 hours, and then left it to cool. The filtrate obtained from filtration was subjected to purification with flash column chromatography on silica gel using mixed solvent of petroleum ether/ethyl acetate (75:25) as eluent. Fractions were monitored by LC/MS. Factions containing component with molecular weight of M=642 were collected, dried, and further purified by recrystallization in mixed solvent of petroleum ether and ethyl acetate to afford 30 mg of pure (±)-3′-O,4′-O-bis(3,4-dimethoxycinnamoyl)-trans-khellactone represented by trans-DMDCK, optical Rotation [α]_{D}=0 (for its Proton Nuclear Magnetic Resonance Spectroscopy (¹H-NMR), see table 1).

### EXAMPLE 4

### Preparation and Structure Identification of (±)-3′-O,4′-O-bis(3,4-dimethoxybenzoyl)-trans-khellactone

(±)-trans-khellactone (80 mg, 0.31 mmol) was dissolved in 5 ml dichloromethane, followed by addition of 3,4-dimethoxybenzoic acid (270 mg, 1.5 mmol), DCC (206 mg, 1 mmol), DMAP (4 mg, 0.032 mmol), reaction was allowed to stir under reflux for 2.5˜3 hours, and then left it to cool. The filtrate obtained from filtration was subjected to purification with flash column chromatography on silica gel using mixed solvent of petroleum ether/ethyl acetate (75:25) as eluent. Fractions were monitored by LC/MS. Factions containing component with molecular weight of M=590 were collected, dried, and further purified by recrystallization in mixed solvent of petroleum ether and ethyl acetate to afford 13 mg of pure (±)-3′-O,4′-O-bis(3,4-dimethoxybenzoyl)-trans-khellactone represented by trans-DMDBK, optical Rotation [α]_{D}=0 (for its Proton Nuclear Magnetic Resonance Spectroscopy (¹H-NMR), see table 1).

TABLE 1
¹H-NMR data (δppm, CDCl₃) of (±)-Praeruptorin A derivatives

| | trans-DMDCK | cis-DMDCK | trans-DMDBK | cis-DMDBK |
|---|---|---|---|---|
| 3-H | 6.22 (1H, d, 9.3 Hz) | 6.20 (1H, d, 9.3 Hz) | 6.19 (1H, d, 9.6 Hz) | 6.14 (1H, d, |
| | | | | 9.4 Hz) |
| 4-H | 7.61 (1H, d, 9.6 Hz) | 7.60 (1H, d, 9.6 Hz) | 7.54 (1H, d, 9.6 Hz) | 7.57 (1H, d, |
| | | | | 9.7 Hz) |
| 5-H | 7.41 (1H, d, 8.4 Hz) | 7.38 (1H, d, 8.5 Hz) | 7.42 (1H, d, 8.1 Hz) | 7.46 (1H, d, |
| | | | | 8.4 Hz) |
| 6-H | 6.86 (1H, d, 8.4 Hz) | 6.85 (1H, d, 8.8 Hz) | 6.80 (1H, d, 8.1 Hz) | 6.85 (1H, d, |
| | | | | 8.5 Hz) |
| 2′-(CH₃)₂ | 1.53, | 1.56, | 1.58, | 1.62, |
| | 1.43 (3H each, s) | 1.46 (3H each, s) | 1.47 (3H each, s) | 1.47 (3H each, s) |
| 3′-H | 5.51 (1H, d, 3.9 Hz) | 5.52 (1H, d, 4.8 Hz) | 5.62 (1H, d, 3.3 Hz) | 5.61 (1H, d, |
| | | | | 4.7 Hz) |
| 4′-H | 6.41 (1H, d, 3.9 Hz) | 6.97 (1H, d, 5.3 Hz) | 6.58 (1H, d, 3.6 Hz) | 6.90 (1H, d, |
| | | | | 4.9 Hz) |
| 2 × (Ar-2-H) | 7.04, | 6.98, | 7.57 (2H, s) | 7.54 (2H, s) |
| | 7.00 (1H each, s) | 6.97 (1H each, s) | | |
| 2 × (Ar-5-H) | 6.82, | 6.76 (2H, d, 8.2 Hz) | 6.90 (2H, d, 8.4 Hz) | 6.87 (2H, d, |
| | 6.81 (1H each, d, 8.4 Hz) | | | 8.5 Hz) |
| 2 × (Ar-6-H) | 7.06 (2H, d, 8.4 Hz) | 6.95 (2H, d, 8.0 Hz) | 7.76 (2H, d, 8.4 Hz) | 7.71 (2H, d, |
| | | | | 8.4 Hz) |
| 4 × (OCH₃) | 3.90, 3.88, 3.87, | 3.88, 3.86, 3.80, | 3.93 (9H, s) | 3.90 (3H, s), |
| | 3.86 (3H each, s) | 3.77 (3H each, s) | 3.88 (3H, s) | 3.89 (3H, s), 3.84 (6H, s) |
| 2 × (Ar—CH═) | 6.30 (1H, d, | 6.31 (2H, d, 15.8 Hz) | | |
| | 15.9 Hz) | | | |
| 2 × (—OCOCH═) | 7.66 (1d, d, 15.9 Hz) | 7.60 (2H, d, 15.5 Hz) | | |

### EXAMPLE 5

### The Effect of Several (±)-Praeruptorin A Derivatives in Reversing Multidrug Resistance in Cancer Cells Caused By Overexpression of P-gp

I. Assay for the Growth Inhibition of Cancer Cell In Vitro

1. Materials and methods

Agents: cis-DMDCK, cis-DMDBK, trans-DMDCK, trans-DMDBK, puromycin, paclitaxel, vinblastine, doxorubicin, and verapamil.

Cells and cell culture: cell lines used in the experiments are human hepatoma cell line (HepG2), human leukemia cell line (K562), human epidermoid carcinoma cell line (KB-3-1) and their multidrug resistant sublines HepG2-DR, K562-DR. and KB V1. The culture conditions for all cells are following: at 37° C. and 5% CO), KB-3-1 and KBV1 were cultured in MEM medium containing 10% fetal bovine serum and 100 U/mL antibiotics; K562, K562-DR, HepG2, HepG2-DR were cultured in RPMI-1640 medium containing 10% fetal bovine serum and 100 U/mL, antibiotics. For maintaining the phenotypic characteristics of multidrug resistance, 1.2 μM and 0.1 μM doxorubicin were respectively added into the mediums of HepG2-DR and K562-DR; 200 ng/mL vinblastine was added into the medium of KBV1. Drug resistant cells were grown in drug free medium for at least 7 days before test.

Drug test: Cell growth inhibitory effects of various drugs were determined by SRB assay in KB-3-1, KB V1, HepG2 and HepG2-DR cells (Skehan P, Storeng R, Scudiero D, Monks A, McMahon J, Vistica D, Warren J T, Bokesch H, Kenney S, Boyd M R. New calorimetric cytotoxicity assay for anticancer-drug screening. J Natl Cancer Inst 1990; 82:1107-12) and by MTT assay in K562 and K562-DR cells (1. Mosmann T. Rapid colorimetric assay for cellular growth and survival: application to proliferation and cytotoxicity assays. J Immunol Methods 1983; 65:55-63. 2. Gerlier D, Thomasset N. Use of MTT calorimetric assay to measure cell activation._{—}J Immunol Methods 1986; 94:57-63), and evaluated by their respective IC₅₀ values (concentration inhibiting 50% of cell growth). Each growth inhibition experiment must be repeated three times and results are expressed as mean±standard deviation (SD). Solvents and media were included as blank control.

In SRB assay, cells are inoculated at 5000 cells/well in a 96-well microplate and incubated overnight to let cells adhere. Drug treatment lasts for 72 hours and cells are fixed for 1 hour at 4° C. with 50 μl ice-cold 15% trichloroacetic acid and washed with triple-distilled water 5 times. Cellular protein is stained by adding 50 μl of 0.4% SRB in 1% acetic acid for 10 minutes, rinsed with 1% acetic acid 5 times and air-dried. The protein-bound dye is dissolved in 100 μl per well of 10 mM Tris base (pH 10.5). The color intensity of SRB, which positively correlate to cell number in preliminary experiments, is estimated at OD 515 nm.

In MTT assay, cells are inoculated at 5000 cells/well and are incubated overnight. Drug treatment lasts for 68 h. MTT (5 mg/ml in PBS) is added to each well (1:10 dilution). After incubation for 4 hour at 37° C., 5% CO₂, 100 μl of stop solution (10% SDS-50% isobutanol-0.01N HCl) is added to each well to stop the reaction. Viable cell number is estimated by correlating to OD at 570 nm.

2. Results

Table 2 and 3 show the growth inhibitory effects of anti-tumor drugs and cis-DMDCK, cis-DMDBK, trans-DMDCK, trans-DMDBK in cancer cells. Compared to parental drug sensitive HepG2, K562 and KB-3-1 cells by IC₅₀ values, drug resistant HepG2-DR, K562-DR and KB V1 cells were highly resistant to the four anticancer drugs tested. The drug resistance ratio (=IC_{50 (drug resistant cell)}/IC_{50 (sensitive cell)}) ranged from 122 to 9271. For example, KB V1 cells were 9271 times more resistant than KB-3-1 cells to vinblastine, HepG2-DR cells were 597 times more resistant than HepG2 cells to puromycin, and K562-DR cells were 5417 times more resistant than K562 cells to pacltaxel (Table 2).

cis-DMDCK, cis-DMDBK, trans-DMDCK, and trans-DMDBK showed no significant growth inhibitory effects (IC₅₀>27 μM) in all six cell lines. Resistance to the four compounds was not observed in drug resistant cells (Table 3).

TABLE 2
Cytotoxity of anti-tumor drugs to tumor cells (IC₅₀, μM)

| Cell line | Vinblastine | Doxorubicin | Puromycin | Paclitaxel |
|---|---|---|---|---|
| KB-3-1 | 0.11 ± 0.01 (×10⁻³) | 0.18 ± 0.04 | 0.21 ± 0.05 | 0.002 ± 0.001 |
| KB V1 | 1.04 ± 0.34 | 31.98 ± 2.74 | 79.30 ± 8.81 | 8.00 ± 1.48 |
| Resistant ratio | 9271 | 178 | 378 | 4000 |
| HepG2 | 0.10 ± 0.01 (×10⁻³) | 0.17 ± 0.10 | 0.17 ± 0.07 | 6.07 ± 2.61 (×10⁻³) |
| HepG2-DR | 0.31 ± 0.06 | 37.27 ± 2.42 | 104.47 ± 2.48 | 4.23 ± 0.06 |
| Resistant ratio | 3100 | 219 | 597 | 696 |
| K562 | 0.88 ± 0.25 (×10⁻³) | 0.24 ± 0.08 | 0.39 ± 0.16 | 0.60 ± 0.23 (×10⁻³) |
| K562-DR | 0.20 ± 0.07 | 31.02 ± 17.07 | 47.69 ± 9.00 | 3.25 ± 0.45 |
| Resistant ratio | 227 | 129 | 122 | 5417 |

TABLE 3
Cytotoxity of (±)-praeruptorin A derivatives to tumor cells (IC₅₀, μM)

| | trans- | | | |
|---|---|---|---|---|
| Cell lines | DMDCK | trans-DMDBK | cis-DMDCK | cis-DMDBK |
| KB-3-1 | 59.64 ± 2.41 | 58.96 ± 0.11 | 61.68 ± 0.63 | 63.94 ± 0.77 |
| KB V1 | 27.71 ± 4.99 | 31.56 ± 2.21 | 32.30 ± 3.35 | 29.48 ± 4.92 |
| Resistant | 0.47 | 0.53 | 0.52 | 0.46 |
| ratio | | | | |
| HepG2 | 55.19 ± 3.62 | 47.51 ± 4.94 | 54.70 ± 3.02 | 49.19 ± 4.34 |
| HepG2- | 75.28 ± 5.70 | 62.35 ± 7.56 | 70.41 ± 10.38 | 58.68 ± 2.07 |
| DR | | | | |
| Resistant | 1.36 | 1.31 | 1.29 | 1.19 |
| ratio | | | | |
| K562 | 62.17 ± 5.63 | 56.52 ± 4.67 | 54.43 ± 4.24 | 65.85 ± 3.47 |
| K562-DR | 88.71 ± 2.01 | 85.14 ± 6.38 | 70.01 ± 3.10 | 60.10 ± 3.29 |
| Resistant | 1.42 | 1.50 | 1.29 | 0.91 |
| ratio | | | | |

II. Assay for the Activity of (±)-Praeruptorin A Derivatives in Reversing Multidrug Resistance in Tumor Cells.

1. Materials and methods: the same with I.

To evaluate the multidrug resistance reversing ability of (±)-Praeruptorin A derivatives, IC₅₀ values of anticancer drugs in the presence and absence of cis-DMDCK, cis-DMDBK, trans-DMDCK, or trans-DMDBK at certain concentration were determined in HepG2-DR, K562-DR and KB V1 cells. The fold decrease of IC₅₀ value of an anticancer drug in certain cell line achieved by a test compound is calculated (fold decrease=IC₅₀ of an anti-tumor drug alone/IC₅₀ of the anti-tumor drug in combination with the test compound) and is used for evaluating the ability of the test compound to reduce drug resistance. The larger the fold decrease value is, the stronger its ability to reverse drug resistance is. The results were average values of three repetitive tests. Verapamil (P-glycoprotein modulator) is the positive control.

2. Results

As shown in table4, all the four compounds significantly reduced drug resistance of drug resistant tumor cells to anti-tumor drugs. cis-DMDCK was the most active. In the presence of 4 μM of cis-DMDCK, IC₅₀ values of vinblastine, doxorubicin, puromycin and paclitaxel in HepG2-DR cells were decreased by 130, 160, 140 and 150 folds, respectively. In the presence of 2 μM of cis-DMDCK, the corresponding decreases were 105, 107, 111 and 89 times, respectively. Even at the concentration as low as 1 μM, cis-DMDCK reduced the relative IC₅₀ values by 45, 22, 29 and 23 times, respectively. cis-DMDBK at 4 μM reduced the drug resistance of HepG2-DR cells to the four anti-tumor drugs by 62-117 times, was the second most active. Similar effects were observed in K562-DR and KB V1 cells. cis-DMDCK exhibited multidrug resistance reversing ability that was obviously superior to the other three compounds. In HepG2-DR or K562-DR cells trans-DMDCK and trans-DMDBK also showed significant effect but were less effective than cis-DMDCK and cis-DMDBK. In KB V1 cells, however, trans-DMDCK and trans-DMDCK exhibited limited ability in reducing drug resistance. For example, decrease in IC₅₀ values of vinblastine or doxorubicin in the presence of 4 μM of trans-DMDCK or trans-DMDCK was less than 5 folds. The ability of the four compounds for reversing drug resistance of tumor cells are listed as following: cis-DMDCK>cis-DMDBK>trans-DMDCK and trans-DMDBK.

TABLE 4
Analysis of the reversing parameter of (±)-Praeruptorin A derivatives

| Cell strains | Drug | | Vinblastine | Doxorubicin | Puromycin | verapamil |
|---|---|---|---|---|---|---|
| HepG2-DR | cis-DMDCK | 4 μM | 129.9 ± 35.5 | 160.6 ± 12.6 | 140.9 ± 65.1 | 159.9 ± 35.0 |
| | | 2 μM | 105.7 ± 21.2 | 107.9 ± 51.9 | 111.1 ± 38.0 | 89.6 ± 24.1 |
| | | 1 μM | 45.7 ± 10.3 | 22.5 ± 9.1 | 29.5 ± 13.0 | 23.1 ± 7.6 |
| | cis-DMDBK | 4 μM | 82.1 ± 13.1 | 117.1 ± 13.8 | 74.6 ± 21.2 | 62.2 ± 7.9 |
| | | 2 μM | 12.4 ± 6.1 | 33.5 ± 7.5 | 15.4 ± 4.6 | 16.1 ± 10.9 |
| | | 1 μM | 4.8 ± 4.1 | 6.2 ± 0.7 | 4.6 ± 1.3 | 6.7 ± 6.4 |
| | trans-DMDCK | 4 μM | 13.9 ± 5.8 | 59.4 ± 18.5 | 26.6 ± 6.4 | 32.9 ± 6.9 |
| | | 2 μM | 1.9 ± 0.0 | 8.0 ± 0.6 | 5.6 ± 0.6 | 1.6 ± 0.6 |
| | trans-DMDBK | 4 μM | 19.3 ± 10.4 | 58.2 ± 17.2 | 28.5 ± 0.2 | 45.3 ± 7.8 |
| | | 2 μM | 1.5 ± 0.3 | 3.8 ± 1.6 | 4.6 ± 1.4 | 1.6 ± 0.6 |
| | varapamil | 4 μM | 4.5 ± 1.5 | 5.7 ± 2.0 | 7.6 ± 1.9 | 4.9 ± 1.3 |
| K562-DR | cis-DMDCK | 4 μM | 138.7 ± 39.6 | 28.8 ± 8.9 | 38.8 ± 8.9 | 169.9 ± 39.4 |
| | cis-DMDBK | 4 μM | 75.6 ± 21.3 | 18.9 ± 7.9 | 21.8 ± 3.1 | 105.8 ± 51.1 |
| | trans-DMDCK | 4 μM | 31.6 ± 5.1 | 16.1 ± 9.6 | 31.1 ± 16.7 | 27.8 ± 8.7 |
| | trans-DMDBK | 4 μM | 11.0 ± 0.6 | 7.0 ± 2.2 | 6.7 ± 0.4 | 9.8 ± 2.1 |
| | verapamil | 4 μM | 7.2 ± 2.6 | 5.2 ± 0.4 | 5.6 ± 0.3 | 31.0 ± 3.3 |
| KB V1 | cis-DMDCK | 4 μM | 331.2 ± 155.4 | 50.0 ± 19.1 | 131.0 ± 7.1 | 499.8 ± 202.8 |
| | | 2 μM | 115.0 ± 17.6 | 19.1 ± 11.1 | 61.4 ± 31.3 | 128.2 ± 53.8 |
| | | 1 μM | 2.3 ± 0.8 | 8.9 ± 2.3 | 37.0 ± 14.4 | 9.0 ± 9.7 |
| | cis-DMDBK | 4 μM | 51.6 ± 11.0 | 16.1 ± 9.5 | 36.8 ± 16.6 | 83.3 ± 35.3 |
| | | 2 μM | 3.5 ± 1.4 | 5.6 ± 2.4 | 15.4 ± 3.3 | 6.8 ± 2.9 |
| | trans-DMDCK | 4 μM | 1.9 ± 0.3 | 3.0 ± 1.2 | 15.3 ± 2.5 | 6.0 ± 3.1 |
| | trans-DMDBK | 4 μM | 1.4 ± 0.2 | 2.7 ± 0.9 | 8.4 ± 4.2 | 2.2 ± 0.4 |
| | verapamil | 4 μM | 3.9 ± 3.0 | 2.3 ± 0.3 | 12.3 ± 4.3 | 9.3 ± 7.4 |

III. The Effect of (±)-Praeruptorin A Derivatives in Recovering the Activity of Doxorubicin for Inducing G2/M arrest in HepG2-DR Cell

1. Materials and methods

1.1 Agents: cis-DMDCK, cis-DMDBK, trans-DMDCK, trans-DMDBK, and doxorubicin.

1.2 Instruments: FACSCAN flow cytometry (Becton Dickinson Immunocytometry Systems, San Jose, Calif.), the obtained data were analyzed using the software of Macintosh CellQuest.

1.3 Cell lines: HepG2, HepG2-DR.

1.4 Drug treatment: HepG2 and HepG2-DR cells were respectively treated with each of cis-DMDCK, cis-DMDBK, trans-DMDCK, trans-DMDBK and doxorubicin for 48 hours, or treated respectively with the combination of doxorubicin and one of cis-DMDCK, cis-DMDBK, trans-DMDCK, trans-DMDBK for 48 hours.

1.5 Treatment and detection of sample cells: after washed twice by ice-cold PBS, the cells were fixed with 70% ethanol at −20° C. overnight. The fixed cells were washed by PBS once and resuspended in 1 mL of PBS containing 100 μg/mL RNAase A and incubated at 37° C. for 30 minutes. Finally, propidium iodide solution (final concentration is 40 μg/mL,) was added to bind with DNA and incubated at room temperature for 5-10 minutes. Cells were analyzed by FACSCAN flow cytometry immediately.

2 Results: Doxorubicin is a topoisomerase II inhibitor that induces G2/M arrest in cell cycle. Table 5 shows that, in drug sensitive HepG2 cells doxorubicin achieved almost a complete G2/M arrest at 0.2 μM but in P-glycoprotein-overexpressing HepG2-DR cells the concentration required was over 50 μM. By themselves cis-DMDCK, cis-DMDBK, trans-DMDCK or trans-DMDBK at 4 μM had no effect on cell cycle of HepG2-DR cell, but significantly enhanced doxorubicin-induced G2/M arrest in HepG2-DR cells. Treatment with 1 μM cis-DMDCK, 2 μM cis-DMDBK, 4 μM trans-DMDCK or 4 μM trans-DMDBK reduced the effective doxorubicin concentration from 50 to 1 μM. These results indicated the four compounds can reverse the dominant drug resistance of multidrug resistant cell to doxorubicin.

TABLE 5
(±)-Praeruptorin A derivatives enhanced
doxorubicin-induced cell cycle arrest in HepG2-DR cells

| | | Cell cycle distribution (%) | | | |
|---|---|---|---|---|---|
| Cells | Drug | SubG₁ | G₀/G₁ | S | G₂/M |
| HepG2 | control | 1.88 ± 1.03 | 60.34 ± 3.59 | 10.06 ± 0.78 | 22.59 ± 0.96 |
| | 0.2 μM doxorubicin | 0.92 ± 0.45 | 4.92 ± 0.91 | 8.89 ± 0.83 | 76.57 ± 1.32 |
| HepG2-DR | control | 1.74 ± 0.14 | 54.91 ± 3.76 | 9.90 ± 4.12 | 32.79 ± 2.11 |
| | 1 μM doxorubicin | 2.37 ± 0.34 | 51.33 ± 0.92 | 7.74 ± 3.57 | 33.47 ± 0.17 |
| | 10 μM doxorubicin | 3.37 ± 0.17 | 24.81 ± 0.23 | 6.86 ± 1.80 | 60.80 ± 0.79 |
| | 50 μM doxorubicin | 4.88 ± 0.75 | 12.05 ± 5.08 | 5.58 ± 1.34 | 73.43 ± 9.00 |
| | 4 μM cis-DMDCK | 2.25 ± 0.51 | 52.10 ± 0.61 | 8.38 ± 4.24 | 33.20 ± 2.05 |
| | 4 μM cis-DMDBK | 2.27 ± 0.49 | 52.36 ± 0.98 | 7.66 ± 3.22 | 34.36 ± 0.42 |
| | 4 μM trans-DMDCK | 2.07 ± 0.06 | 47.46 ± 0.55 | 7.97 ± 1.05 | 33.42 ± 1.98 |
| | 4 μM trans-DMDBK | 1.33 ± 0.24 | 49.57 ± 0.15 | 6.17 ± 1.55 | 34.41 ± 3.70 |
| | 1 μM doxorubicin + | 4.35 ± 0.59 | 8.46 ± 1.63 | 6.52 ± 1.76 | 79.79 ± 4.56 |
| | 0.5 μM cis-DMDCK | | | | |
| | 1 μM cis-DMDCK | 5.59 ± 4.35 | 4.39 ± 1.50 | 5.36 ± 3.13 | 81.42 ± 6.02 |
| | 2 μM cis-DMDCK | 6.40 ± 5.54 | 4.51 ± 0.45 | 8.87 ± 4.04 | 77.12 ± 3.36 |
| | 0.5 μM cis-DMDBK | 2.92 ± 1.09 | 35.82 ± 2.26 | 6.44 ± 0.77 | 52.79 ± 5.64 |
| | 1 μM cis-DMDBK | 4.08 ± 0.81 | 13.52 ± 1.36 | 5.85 ± 3.09 | 73.03 ± 1.89 |
| | 2 μM cis-DMDBK | 7.13 ± 6.57 | 3.74 ± 0.76 | 6.86 ± 2.57 | 79.01 ± 6.01 |
| | 1 μM trans-DMDCK | 3.94 ± 0.77 | 34.29 ± 0.21 | 8.80 ± 3.64 | 48.46 ± 2.52 |
| | 2 μM trans-DMDCK | 6.55 ± 0.12 | 21.21 ± 1.48 | 5.79 ± 4.05 | 64.06 ± 3.56 |
| | 4 μM trans-DMDCK | 4.29 ± 1.99 | 6.03 ± 0.91 | 5.70 ± 4.20 | 79.52 ± 0.66 |
| | 1 μM trans-DMDBK | 3.31 ± 0.89 | 34.60 ± 2.58 | 7.61 ± 3.69 | 49.53 ± 0.69 |
| | 2 μM trans-DMDBK | 5.30 ± 2.52 | 17.56 ± 2.20 | 7.03 ± 2.34 | 67.89 ± 1.19 |
| | 4 μM trans-DMDBK | 5.11 ± 2.84 | 5.37 ± 1.06 | 4.71 ± 3.62 | 81.56 ± 2.62 |

### EXAMPLE 6

### The Effect of (±)-Praeruptorin A Derivatives on the Transport Ability of P-gp in Multidrug Resistant Cells

I. Doxorubicin Accumulation Assay

1. Materials and methods

Agents: cis-DMDCK, cis-DMDBK, trans-DMDCK, trans-DMDBK, doxorubicin and verapamil (as positive control)

Cell lines: K562-DR, HepG2-DR

Instruments: as described in example 5 III 1.2

Test for accumulation level of doxorubicin: About 1×10⁶ HepG2-DR or K562-DR cells were suspended in 1 ml of medium containing 10 μM doxorubicin with or without 2 μM, 5 μM or 10 μM cis-DMDCK, cis-DMDBK, trans-DMDCK, trans-DMDBK or verapamil and incubated at 37° C. for 1 hour. Cells were washed with ice-cold PBS twice and resuspended in 1 ml of ice-cold PBS. Cellular doxorubicin fluorescent intensity was monitored by a FACSCAN flow cytometer (Becton Dickinson Immunocytometry Systems, San Jose, Calif., USA). Data were analyzed with the Macintosh CellQuest software. In the experiment, verapamil, the known P-gp inhibitor, was used as the positive control.

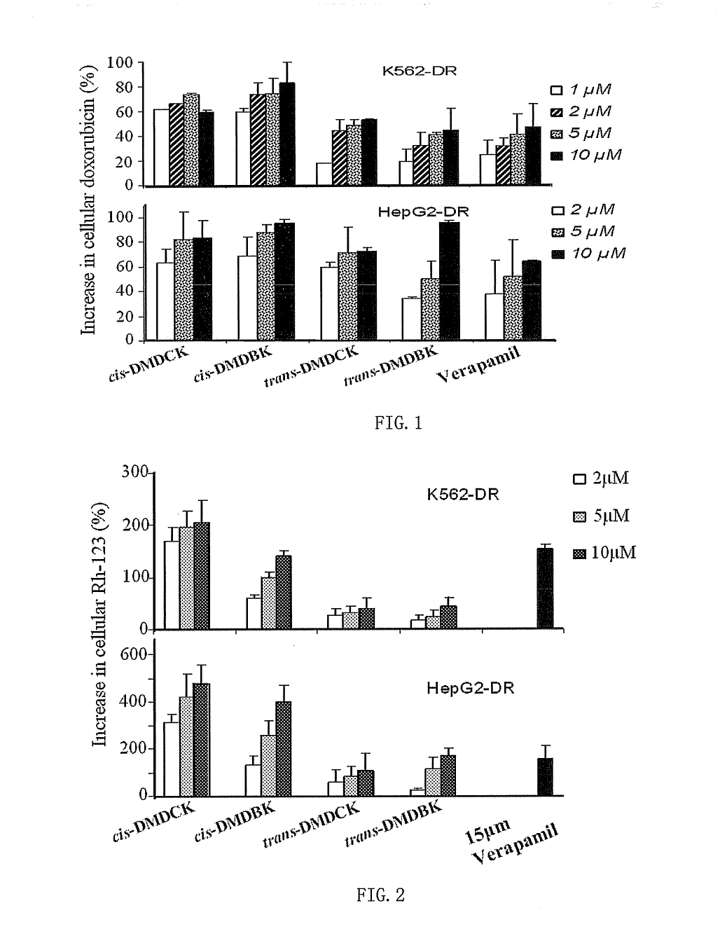

2. Results: Due to Pgp overexpression, drug concentration in tumor cells could not reach the desired effective level, so the effect thereof was lessoned and thus causing the cells possess drug resistance. In this study, cis-DMDCK, cis-DMDBK, trans-DMDCK, trans-DMDBK or verapamil significantly increased cellular doxorubicin accumulation within HepG2-DR and K562-DR cells in a dose-dependent manner. Consistent with their activity in reversing multidrug resistance, cis-DMDCK and cis-DMDBK were more active than trans-DMDCK and trans-DMDBK. With addition of 5 μM cis-DMDCK or cis-DMDBK, cellular doxorubicin fluorescence increased by more than 80% in HepG2-DR and by more than 70% in K562-DR cells, compared to 50% and 40% of increases induced by 5 μM verapamil. trans-DMDCK also exhibited higher activity than verapamil in both HepG2-DR and K562-DR cells. trans-DMDBK showed comparative activity to verapamil at lower concentration but at 10 μM it exhibited higher activity in HepG2-DR cell than verapamil (shown in FIG. 1).

II. Rhodamine-123 Efflux Assay

1. Materials and methods

1.1 Agents: cis-DMDCK, cis-DMDBK, trans-DMDCK, trans-DMDBK, rhodamine-123 (Rh-123), and verapamil (positive control)

1.2 Cell line: as described in example 5 I 1.2

1.3 Instruments: as described in example 5 III 1.2

1.4 Test for Rh-123 transport: HepG2-DR cells or K562-DR cells (1×10⁶ cells in 1 mL complete growth medium) were incubated with 5 μg/mL, Rh-123 at 37° C. for 1 hour to allow Rh-123 uptake. Rh-123 loaded cells were washed with ice-cold PBS twice, and resuspended in 1 mL, fresh medium with or without various concentrations of cis-DMDCK, cis-DMDBK, trans-DMDCK, trans-DMDBK or verapamil. After 1 hour of incubation at 37° C., cells were washed with ice-cold PBS twice and resuspended in 1 mL ice-cold PBS. Cellular fluorescence of Rh-123 was determined by flow cytometry to analyze the inhibitive effect of test compounds on drug expulsion from cells. In the experiments, verapamil, the known P-gp inhibitor, was used as the positive control.

2. Results: Rh-123 is a fluorescent P-gp substrate. Due to the transport activity of P-gp, the Rh-123 level in HepG2-DR and K562-DR cell decreased dramatically one hour after the dye was removed from the medium. The experimental results indicated cis-DMDCK, cis-DMDBK, trans-DMDCK and trans-DMDBK had the ability to slow down the Rh-123 loss in P-gp overexpressed tumor cell. Among them, cis-DMDCK and cis-DMDBK had the most significant effect. Compared with untreated cells, treatment with 10 μM cis-DMDCK or cis-DMDBK caused 480% or 400% increases in cellular Rh-123 fluorescence in HepG2-DR cells, and 200% or 140% increases in K562-DR cells, respectively. Corresponding increases in cellular Rh-123 fluorescence caused by 10 μM trans-DMDCK or trans-DMDBK were less than 200% and 50%, respectively (FIG. 2).

III. Hoechst 33342 Efflux Assay

1. Materials and methods

1.1 Agents: cis-DMDCK, cis-DMDBK, trans-DMDCK, trans-DMDBK, and Hoechst 33342

1.2 Cell line: HepG2-DR

1.3 Instruments: BMG FLUOstar OPTIMA Microplate Reader

1.4 Hoechst 33342 transport test: HepG2-DR Cells (5×10⁴ cells in 100 μL, medium per well) were seeded in 96-well plate and incubated overnight to permit cell attachment. The medium was replaced with fresh medium containing 20 μg/mL Hoechst 33342 and cells were incubated at 37° C. for 1 hour. Cells were then washed with 100 μL ice-cold PBS twice. New medium containing the test compound of various concentrations was added and cells were further incubated at 37° C. for 1 hour. Cells were washed with ice-cold PBS twice and cellular fluorescence intensity was measured at λₑₓ=365 nm (λₑₘ=460 nm) by a BMG FLUOstar OPTIMA Microplate Reader. Inhibitory effect of the test compound on Hoechst 33342 efflux was expressed as the percentage increase of retained Hoechst 33342 in cells.

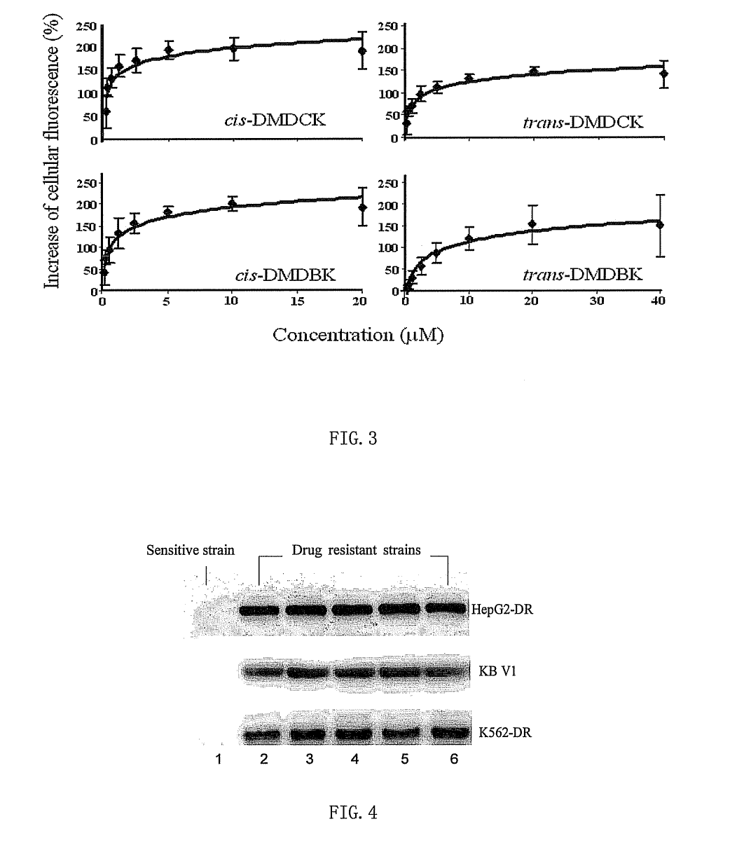

2. Results: Hoechst 33342 is another fluorogenic substrate of P-gp, and acts on a binding site different from Rh-123. The experimental results indicated cis-DMDCK, cis-DMDBK, trans-DMDCK and trans-DMDBK could slow down the Hoechst 33342 loss in HepG2-DR cell in a dose-dependent manner (FIG. 3). cis-DMDCK and cis-DMDBK had the most significant effect. Compared with untreated cells, 5 μM cis-DMDCK or 10 μM cis-DMDBK achieved the highest effect of 200% increase of cellular Hoechst 33342 fluorescence. For trans-DMDCK and trans-DMDBK at 20 μM and the highest increase of cellular Hoechst 33342 fluorescence was about 150%. Similarly, the inhibitive effects of the four compounds on the Hoechst 33342 efflux out of cells are as follows: cis-DMDCK>cis-DMDBK>trans-DMDCK and trans-DMDBK (shown in FIG. 3).

### EXAMPLE 7

### Assay for the Interaction Between (±)-Praeruptorin A Derivatives and P-gp

I. Effect of (±)-Praeruptorin A Derivatives on the Expression of P-gp in Drug Resistant Cell

1. Materials and methods

Agents: cis-DMDCK, cis-DMDBK, trans-DMDCK, trans-DMDBK

Cell lines: as described in example 5 I 1.2.

Immunoblotting analysis of P-gp expression: cells were treated with 4 μM of cis-DMDCK, 4 μM of cis-DMDBK, 4 μM of trans-DMDCK, or 4 μM of trans-DMDBK for 72 hours. Treated cells were collected and mixed well in ice-cold lysis buffer (50 mM Tris pH7.4, 100 mM NaCl, 2 mM EDTA, 1% sodium deoxycholate, 0.1% SDS, 1% triton X-100, 2 mM PMSF, 1% aprotinin) for 30 minutes and then centrifuged to get the total protein. Protein concentration was determined using Bradford assay. In this experiment, 50 μg total protein was separated by 8% SDS-PAGE and electro-transferred to nitrocellulose membranes. The membrane was blocked by 5% skim milk/0.1% Tween-20/TBS (10 mM Tris pH7.5, 100 mM NaCl), and then incubated with anti-P-pg antibody for 1 hour, followed by horseradish-peroxidase-conjugated secondary antibody for another 1 hour. Protein bands were detected by the ECL method.

2. Results: Experimental results were shown in FIG. 4, KB V1, HepG2-DR and K562-DR cell expressed P-gp at high level when compared with parental drug sensitive cells thereof. After a 72 hour treatment with 4 μM cis-DMDCK, cis-DMDBK, trans-DMDCK, or trans-DMDBK, there was no detectable alteration on the expression level of MDR1 in all the three cell lines.

II. Assay for the Effect of (±)-Praeruptorin A Derivatives on P-gp Reactivity to Monoclonal Antibody UIC2 (MDR1 Reactivity Shift Assay).

1. Materials and methods

1.1 Agents: cis-DMDCK, cis-DMDBK, trans-DMDCK, trans-DMDBK, sodium vanadate, and cyclosporine A

1.2 Cell line: HepG2-DR

1.3 Instruments: as described in example 5 III 1.2.

1.4 MDR1 reactivity shift assay: HepG2-DR Cells were washed with PBS and resuspended in UIC2 binding buffer (PBS+1% BSA). Approximately 1×10⁶ cells in 1 mL, UIC2 binding buffer (1% BSA PBS solution) were pre-warmed at 37° C. for 10 minutes, incubated with drugs at 37° C. for another 10 minutes, and 1 μg of the monoclonal antibody UIC2 was added. After 15 minutes at 37° C., 700 μL, of ice-cold UIC2 buffer was added to stop the reaction. Cell samples were washed with ice-cold UIC2 binding buffer twice, resuspended in 500 μL ice-cold UIC2 binding buffer and 2 μL, of goat anti-mouse 1 gC₂ₐ-PE was added. After 15 minutes at 4° C. in the dark, samples were washed, resuspended in 1 ml ice-cold UIC2 binding buffer and analyzed by using a FACSCalibur flow cytometer.

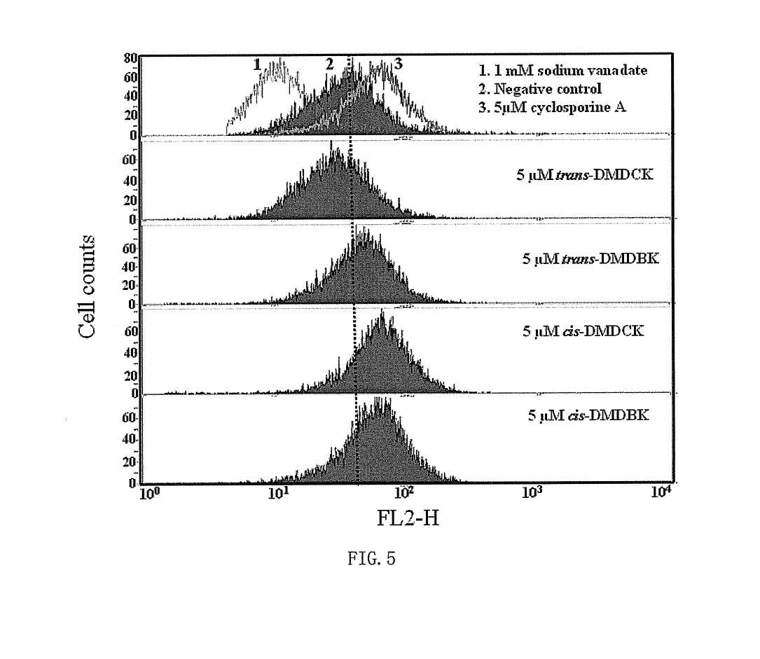

2. Results: Conformation-sensitive monoclonal antibody UIC2 preferentially recognizes Pgp that is associated with transport substrate or competitive inhibitors (1. Mechetner E B, Schott B, Morse B S, Stein W D, Druley T, Davis K A, Tsurtio T, Roninson I B. P-glycoprotein function involves conformational transitions detectable by differential immunoreactivity. Proc Natl Acad Sci USA 1997; 94:12908-12913. 2. Nagy H, Goda K, Arceci R, Cianfriglia M, Mechetner E, Szabo G J. P-Glycoprotein conformational changes detected by antibody competition. Eur J Biochem 2001; 268: 2416. 3. Maki N, Hafkemeyer P, Dey S. Allosteric modulation of human P-glycoprotein. Inhibition of transport by preventing substrate translocation and dissociation. J Biol Chem 2003; 278:18132-18139). P-gp conformational change caused by binding of a substrate can increase UIC2 reactivity to Pgp whereas the conformational change caused by binding of a compound to the allosteric site on Pgp can decrease UIC2 reactivity. Thus, UIC2 reactivity indirectly reflects the drug-Pgp interaction. UIC2 binding can be detected by labeling with a fluorescent secondary antibody. The intensity of cellular fluorescence positively reflects the reactivity of UIC2 with P-gp. In this experiment, P-gp substrate control cyclosporine A increased cellular fluorescence whereas sodium vanadate, an allosteric modulator of P-gp, decreased the cellular fluorescence. 5 μM cis-DMDCK, cis-DMDBK and trans-DMDBK increased cellular fluorescence like cyclosporine A, showed substrate-like activity. 5 μM trans-DMDCK decreased cellular fluorescence like sodium vanadate, implying an interaction between trans-DMDCK and the allosteric site on Pgp (shown in FIG. 5).

1. 3′,4′-aromatic acyloxy substituted 7,8-pyranocoumarin compounds having the following general formula: 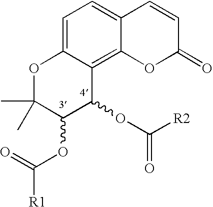
wherein, R1 and R2 may be the same or different, independently represents alkoxy-substituted aryl, and ester groups at C-3′ and C-4′ are either in cis-configuration or trans-configuration; said cis-configuration may be 3′(R),4′(R) or 3′(S),4′(S) or combination of these two; trains-configuration may be 3′(R),4′(S) or 3′(S),4′(R) or combination of these two.

2. The compounds according to claim 1, wherein said alkoxy-substituted aryl is selected from methoxy-substituted aryl.

3. The compounds according to claim 2, wherein said methoxy-substituted aryl is selected from group consisting of 4-methoxyphenyl, 4-methoxybenzyl, 4-methoxystyryl, 3,4-dimethoxyphenyl, 3,4-dimethoxybenzyl and 3,4-dimethoxystyryl.

4. The compounds according to claim 2, wherein said compound is (±)-3′-O,4′-O-bis(3,4-dimethoxybenzoyl)-cis-khellactone, (±)-3′-O,4′-O-bis(3,4-dimethoxybenzoyl)-trans-khellactone, (±)-3′-O,4′-O-bis(3,4-dimethoxycinnamoyl)-cis-khellactone, or (±)-3′-O,4′-O-bis(3,4-dimethoxycinnamoyl)-trans-khellactone.

5. A process for preparation of the compounds according to claim 1 comprising the following steps:
(a) To prepare (±)-3′,4′-cis-dihydoxy 7,8-pyranocoumarin or (±)-3′,4′-trans-dihydoxy 7,8-pyranocoumarin respectively using (±)-Praeruptorin A as the lead compound; and
(b) To prepare (±)-3′,4′-cis-diaromatic acyloxy substituted 7,8-pyranocoumarins or (±)-3′,4′-trans-diaromatic acyloxy substituted 7,8-pyranocoumarins from (±)-3′,4′-cis-dihydoxy 7,8-pyranocoumarin or (±)-3′,4′-trans-dihydoxy 7,8-pyranocoumarin, respectively.

6. The process according to claim 5, wherein said steps (a) and (b) are as follows:
(a) To dissolve (±)-Praeruptorin A in dioxane and stir it at about 60° C. for 10˜30 minutes in the presence of about 0.5 M potassium hydroxide, followed by slow addition of about 10% sulphuric acid at room temperature for acidification, and then extract the resultant reaction solution with chloroform, and undergo purification with silica gel column chromatography to afford (±)-3′,4′-cis-dihydoxy 7,8-pyranocoumarin and (±)-3′,4′-trans-dihydoxy 7,8-pyranocoumarin, respectively; and
(b) To dissolve (±)-3′,4′-cis-dihydoxy 7,8-pyranocoumarin or (±)-3′,4′-trans-dihydoxy 7,8-pyranocoumarin in dichloromethane, respectively, followed by stirring under reflux with 5˜8 times by mole of aromatic carboxylic acid compound in the presence of dicyclohexylcarbodiimide and 4-dimethylamino pyridine for 2.5˜3 hours. Filter the resultant reaction solution after cooling, and the filtrate is subjected to purification with silica gel column chromatography to afford pure (±)-3′,4′-cis-diaromatic acyloxy substituted 7,8-pyranocoumarins and (±)-3′,4′-trans-diaromatic acyloxy substituted 7,8-pyranocoumarins, respectively.

7. A pharmaceutical composition comprising the compound of any one of claims 1˜7.

8. The pharmaceutical composition according to claim 7, wherein said pharmaceutical composition is in the form of parenteral preparations or oral preparations.

9. The pharmaceutical composition according to claim 8, wherein said parenteral preparations are injection preparations.

10. The pharmaceutical composition according to claim 8, wherein said oral preparations are selected from the group consisting of tablets, capsules, granules and oral solution.

11. A method for treating cancers comprising the step of administering therapeutically effective amount of the compound of any one of claims 1˜7 or pharmaceutical composition of any one of claims 10˜13 to a subject in need thereof.

12. The method according to claim 11, wherein said compound or pharmaceutical composition possess the following efficacies:
(a) Increasing the sensitivity of multidrug-resistant cancer cells to anti-cancer medicaments;
(b) Reactivating Doxorubicin in drug-resistant cells to induce G2/M arrest, which lead to cell apoptosis;
(c) Significantly increasing the accumulation level of Doxorubicin in drug-resistant cells; or
(d) Significantly decreasing the expulsion of Rh-123 and H33342 in drug-resistant cells.

13. The method according to claim 11, wherein the compound or the pharmaceutical composition may be administered in combination with an anticancer medicament.

14. The method according to claim 13, wherein the anti-cancer medicament is selected from the group consisting of Doxorubicin, Vinblastine, Puromycin and Paclitaxel.
